(19) United States
(12) Patent Application Publication
(10) Pub. No.: US 2008/0081354 A1
(43) Pub. Date: Apr. 3, 2008

(54) Devices, vectors and methods for inducible ischemia cardioprotection

(75) Inventors: Jihong Qu, Maple Grove, MN (US); Andrew P. Kramer, Stillwater, MN (US); Stephen Ruble, Lino Lakes, MN (US)

Correspondence Address: SCHWEGMAN, LUNDBERG & WOESSNER, P.A., P.O. BOX 2938, MINNEAPOLIS, MN, 55402, US

(73) Assignee: Cardiac Pacemakers, Inc.

(21) Appl. No.: 11/541,757

(22) Filed: Oct. 2, 2006

(51) Int. Cl. C12P 21/06; C07H 21/04; C07K 14/705; C12N 5/08

(52) U.S. Cl. 435/69.1; 435/320.1; 435/325; 435/366; 530/350; 536/23.5

(57) ABSTRACT

Vectors, systems having implantable devices and methods useful to detect or treat ischemia are provided.

### BACKGROUND

The heart is the center of a person's circulatory system. It includes an electromechanical system performing two major pumping functions. The left portions of the heart draw oxygenated blood from the lungs and pump it to the organs of the body to provide the organs with their metabolic needs for oxygen. The right portions of the heart draw deoxygenated blood from the organs and pump it into the lungs where the blood gets oxygenated. The body's metabolic need for oxygen increases with the body's physical activity level. The pumping functions are accomplished by contractions of the myocardium (heart muscles). In a normal heart, the sinoatrial node, the heart's natural pacemaker, generates electrical impulses, known as action potentials, that propagate through an electrical conduction system to various regions of the heart to excite myocardial tissues in these regions. Coordinated delays in the propagations of the action potentials in a normal electrical conduction system cause the various regions of the heart to contract in synchrony such that the pumping functions are performed efficiently.

A blocked or otherwise damaged electrical conduction system causes the myocardium to contract at a rhythm that is too slow, too fast, irregular or dyssynchronous. Such an abnormal rhythm is generally known as arrhythmia. Arrhythmia reduces the heart's pumping effectiveness and hence, diminishes the blood flow to the body. A deteriorated myocardium has decreased contractility, also resulting in diminished blood flow. A heart failure patient usually suffers from both a damaged electrical conduction system and a deteriorated myocardium. The diminished blood flow results in insufficient blood supply to various body organs, preventing these organs to function properly and causing various symptoms. For example, in a patient suffering acute decompensated heart failure, an insufficient blood supply to the kidneys results in fluid retention and edema in the lungs and peripheral parts of the body, a condition referred to as decompensation. Without effective treatment, acute decompensated heart failure cause rapid deterioration of the cardiovascular and general health and significantly shortened life expectancy. Treatments for arrhythmias and heart failure include, but are not limited to, electrical therapy such as pacing and defibrillation therapies, drug therapies, and biological therapies including gene-based therapies.

Gene-based therapies include the delivery of therapeutic genes to targeted cells and in some cases, the use of regulatable systems. For gene-based therapies which require expression of sequences in vectors, a promoter is linked to the sequence to be expressed. Strong viral promoters can drive a high level of expression in a wide range of tissues and cells, however, constitutive expression is an open loop system and the encoded gene product may induce cellular toxicity or tolerance, or down regulation of expression through negative feedback.

One strategy to regulate the expression of target genes employs endogenous regulatable elements, and another strategy employs exogenous inducible gene expression systems. For example, heat-shock-induced loci have been used to regulate the expression of a heterologous gene in mammalian cells (Wurm et al., Proc. Natl. Acad. Sci. USA, 83:5414 (1986); Bovenberg et al., Mol. Cell Endocrinol., 74:45 (1990)), and hypoxia-inducible cis-acting sequences from the erythropoietin gene allow a transcriptional response by hypoxia-inducible transcription factor (HIF-I) (Wang et al., Curr. Op. Hematol., 3:156 (1996)). However, many regulatable systems based on endogenous promoters suffer from weak induction and high basal expression.

What is needed is a rapid diagnostic system for an ischemic event and a therapy that is automatically triggered by detection of an ischemic event.

### SUMMARY OF THE INVENTION

The invention provides vectors, methods and systems to rapidly detect ischemia, such as myocardial ischemia (e.g., acute myocardial infarction) or ischemia associated with heart failure, and optionally provide a treatment including expression of a cardioprotective or therapeutic gene product, e.g., a gene product that can rescue or protect cells, e.g., in the myocardium, which cells are at risk of ischemic damage. The gene therapy may optionally be combined with electrical therapy (e.g., a vagal stimulation therapy (VST) in which neurostimulation is delivered to the vagus nerve) or drug therapy. The vectors, methods and systems employ a hypoxia stable gene product and a recombinant gene regulated thereby, such as one induced by a hypoxia inducible factor (HIF), e.g., HIF1α, or cyclic AMP response element binding (CREB) protein. The recombinant gene has a specific DNA sequence in its transcriptional regulatory region which binds a hypoxia stable gene product. The use of such a recombinant gene in vivo provides specific location information for the ischemia, e.g., useful in diagnostics, or may provide induction of a gene therapy at a particular location subsequent to, e.g., immediately after, an ischemic event.

In one embodiment, a vector system is employed to detect and/or treat disease with high specificity and rapid action. The vector system may include a first vector comprising a transcriptional regulatory region having an organ-specific, tissue-specific or cell-specific transcriptional regulatory element operably linked to a first open reading frame for a gene product (“an expression cassette”). In another embodiment, the vector system may include a first vector comprising a transcriptional regulatory region which is regulated by energy, e.g., by electrical pacing, light or a magnetic field, operably linked to a first open reading frame for a gene product. In one embodiment, the energy-regulated element is an inducible element. The gene product is unstable under normoxic conditions, stable under hypoxic conditions and binds a specific DNA sequence. In one embodiment, the promoter in the transcriptional regulatory region is constitutively expressed. The organ-, tissue-, or cell-specific, or energy-regulated, transcriptional regulatory element may be a promoter. The vector system also includes a second vector comprising a transcriptional regulatory region comprising the specific DNA sequence operably linked to a second opening reading frame for a therapeutic gene product, a cardioprotective gene product or a biomarker. As a result of an organ-, tissue- or cell-specific, or energy-regulated, transcriptional regulatory element, e.g., one expressed in the heart or vasculature, detection of the ischemia event, detection of the location of the ischemic event, gene therapy for the ischemia in that organ, tissue or cell, or any combination thereof, may be achieved. Further, the detection, and optionally the location of the ischemic event or treatment thereof, only occurs under particular pathophysiological states, e.g., ischemia or in diabetics. For instance, the ability to detect ischemia in patients that experience considerable variation in blood glucose levels provides for detection and/or treatment of ischemic damage due to diabetes. In one embodiment, the vector system is employed to inhibit or treat cardiac remodeling associated with heart failure, e.g., dilated cardiomyopathy, as there is a subnormal level of oxygen available to the myocardium in heart failure patients, e.g., an ischemia in the endocardial layers. In another embodiment, the vector system is employed to inhibit or treat chronic heart failure, e.g., to prevent or reverse cardiac remodeling and/or dilatation. In one embodiment, the transcriptional regulatory region in the second vector having a specific DNA sequence for the normoxic unstable and hypoxia stable gene product increases expression of a linked open reading frame of interest, for example, in about 1 to 2 hours after an ischemic event, and so is useful to rapidly detect ischemia, and optionally provide for cardioprotection after myocardial infarction. Thus, the vector system allows for rapid detection of ischemia and optionally automatic increased expression of an open reading frame of interest in a selective manner, for instance, at the ischemic site(s), e.g., the heart. The vectors, methods and systems of the invention are applicable to diagnosing, or preventing, inhibiting or treating, ischemia in any organ or tissue, and may be used with revascularization procedures or other therapies to protect the heart (or other organs or tissues) from ischemia-reperfusion injury. In one embodiment, the system combines the power of genes and implantable devices to diagnose, or inhibit or treat, cardiac ischemia.

The present invention thus provides spatial and temporal detection and optionally treatment of cells in vivo in response to ischemic conditions. In one embodiment, one of the vectors in the vector system of the invention encodes one or more gene products useful to inhibit ischemic damage in a cell having the vector system and/or in adjacent cells, e.g., via intercellular channels, or by secretion or other mechanisms that result in the release of the desired gene product into the extracellular space.

The invention also provides an isolated (exogenous) mammalian cell having the vector system. In one embodiment, the mammalian cell is a stem cell. In another embodiment, the mammalian cell is an autologous cell. In one embodiment, the mammalian cell may be introduced to a host mammalian organism, e.g., via injection or an implantable device.

The invention further provides for a system having a composition comprising at least two vectors, one vector has a transcriptional regulatory region which includes an organ-, tissue- or cell-specific, or an energy-regulated, transcriptional regulatory element operably linked to a first open reading frame for a gene product that is unstable under normoxic conditions, stable under hypoxic conditions and binds a specific DNA sequence. Another vector has a transcriptional regulatory region having the specific DNA sequence linked to a second opening reading frame for a therapeutic or cardioprotective gene product, and an implantable device for electrical or drug therapy. The composition may form part of or be delivered by a device, e.g., an implantable gene delivery device, or a percutaneous catheter with or without an injection needle. In one embodiment, pacing pulses from an implantable device along with expression of a therapeutic or cardioprotective gene product provide for beneficial effects, e.g., effects which are additive or synergistic in a mammal having the system and the device.

Also provided are methods of using the vectors. In one embodiment, the vector system of the invention is introduced to a mammal, e.g., a mammal at risk of cardiac ischemia, where one of the vectors encodes a therapeutic or cardioprotective gene product. The expression of the gene product in the mammal in an effective amount inhibits or treats ischemia. In one embodiment, the mammal is also subjected to electrical therapy, e.g., pacing. In another embodiment, the vector system of the invention is introduced to a mammal, where one of the vectors encodes a biomarker, such as a secretable biomarker or a cell associated protein such as an anchor protein. In one embodiment, the presence or location of the biomarker or cell associated protein in the mammal is detected. For example, the presence of a secreted biomarker may be detected in a physiological fluid or the location of a cell associated protein such as an anchor protein may be detected after administering a radioactive ligand for the protein.

In one embodiment, the vectors are introduced (administered) to a mammal is at risk of ischemia. In another embodiment, the vectors are introduced to a mammal having or at risk of heart failure. In another embodiment, the vectors are introduced to a mammal having or at risk of coronary artery disease, vulnerable plaque or stroke. In one embodiment, the vectors that are introduced to the mammal are on the same molecule, e.g., DNA molecule such as a plasmid. In one embodiment, the vectors are introduced into exogenous donor cells, e.g., autologous donor stem cells, prior to introduction to the mammal. In one embodiment, the vectors are introduced to the mammal via intracardiac or intravenous administration. In one embodiment, the vectors are introduced to a tissue or organ, e.g., to the heart. For instance, the vectors may be injected into a tissue or organ. In another embodiment, a mammal having the vectors also is subjected to electrical stimulation, e.g., neurostimulation or pacing pulses, administered a a drug, or a combination thereof. In one embodiment, an implantable device delivers electrical stimulation or one or more drugs, or a combinantion thereof, to a mammal having the vectors.

### BRIEF DESCRIPTION OF THE FIGURES

FIG. 1 is a schematic of an exemplary vector system useful in the systems and methods of the invention.

FIG. 2 is an illustration of combined vector system and device therapy.

FIG. 3 is a block diagram showing an embodiment of a cardioprotective device for delivering the device therapy.

FIG. 4 is a block diagram showing a specific embodiment of the cardioprotective device for delivering the device therapy.

FIG. 5 is an illustration of an embodiment of a system including the cardioprotective device and portions of an environment in which the system operates.

### DETAILED DESCRIPTION OF THE INVENTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized and that structural, logical and electrical changes may be made without departing from the spirit and scope of the present invention. The following detailed description provides examples, and the scope of the present invention is defined by the appended claims and their equivalents.

It should be noted that references to “an”, “one”, or “various” embodiments in this disclosure are not necessarily to the same embodiment, and such references contemplate more than one embodiment. Definitions A “vector” or “construct” (sometimes referred to as gene delivery or gene transfer “vehicle”) refers to a macromolecule or complex of molecules comprising a polynucleotide to be delivered to a host cell, either in vitro or in vivo. The polynucleotide to be delivered may comprise a sequence of interest for gene therapy. Vectors include, for example, transposons and other site-specific mobile elements, viral vectors, e.g., adenovirus, adeno-associated virus (AAV), poxvirus, papillomavirus, lentivirus, herpesvirus, foamivirus and retrovirus vectors, and including pseudotyped viruses, liposomes and other lipid-containing complexes, and other macromolecular complexes capable of mediating delivery of a polynucleotide to a host cell, e.g., DNA coated gold particles, polymer-DNA complexes, liposome-DNA complexes, liposome-polymer-DNA complexes, virus-polymer-DNA complexes, e.g., adenovirus-polylysine-DNA complexes, and antibody-DNA complexes. Vectors can also comprise other components or functionalities that further modulate gene delivery and/or gene expression, or that otherwise provide beneficial properties to the cells to which the vectors will be introduced. Such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. A large variety of such vectors are known in the art and are generally available. When a vector is maintained in a host cell, the vector can either be stably replicated by the cells during mitosis as an autonomous structure, incorporated within the genome of the host cell, or maintained in the host cell's nucleus or cytoplasm.

A “recombinant viral vector” refers to a viral vector comprising one or more heterologous genes or sequences. Since many viral vectors exhibit size constraints associated with packaging, the heterologous genes or sequences are typically introduced by replacing one or more portions of the viral genome. Such viruses may become replication-defective, requiring the deleted function(s) to be provided in trans during viral replication and encapsidation (by using, e.g., a helper virus or a packaging cell line carrying genes necessary for replication and/or encapsidation). Modified viral vectors in which a polynucleotide to be delivered is carried on the outside of the viral particle have also been described (see, e.g., Curiel et al., Proc. Natl. Acad. Sci. USA, 88:8850 (1991)).

“Gene delivery,” “gene transfer,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, iontophoresis, “gene gun” delivery, or via extracellular matrix or hydrogel scaffolding, and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art.

By “transgene” is meant any piece of a nucleic acid molecule (for example, DNA) which is inserted by artifice into a cell either transiently or permanently, and becomes part of the organism if integrated into the genome or maintained extrachromosomally. Such a transgene may include a gene which is partly or entirely heterologous (i.e., foreign) to the transgenic organism, or may represent a gene homologous to an endogenous gene of the organism.

By “transgenic cell” is meant a cell containing a transgene. For example, a stem cell transformed with a vector containing an expression cassette can be used to produce a population of cells having altered phenotypic characteristics.

The term “wild-type” refers to a gene or gene product that has the characteristics of that gene or gene product when isolated from a naturally occurring source. A wild-type gene is that which is most frequently observed in a population and is thus arbitrarily designated the “normal” or “wild-type” form of the gene. In contrast, the term “modified” or “mutant” refers to a gene or gene product that displays modifications in sequence and or functional properties (i.e., altered characteristics) when compared to the wild-type gene or gene product. It is noted that naturally-occurring mutants can be isolated; these are identified by the fact that they have altered characteristics when compared to the wild-type gene or gene product.

“Vasculature” or “vascular” are terms referring to the system of vessels carrying blood (as well as lymph fluids) throughout the mammalian body.

“Blood vessel” refers to any of the vessels of the mammalian vascular system, including arteries, arterioles, capillaries, venules, veins, sinuses, and vasa vasorum.

“Artery” refers to a blood vessel through which blood passes away from the heart. Coronary arteries supply the tissues of the heart itself, while other arteries supply the remaining organs of the body. The general structure of an artery consists of a lumen surrounded by a multi-layered arterial wall.

The term “transduction” denotes the delivery of a polynucleotide to a recipient cell either in vivo or in vitro, via a viral vector and preferably via a replication-defective viral vector, such as via a recombinant AAV.

The term “heterologous” as it relates to nucleic acid sequences such as gene sequences and control sequences, denotes sequences that are not normally joined together, and/or are not normally associated with a particular cell. Thus, a “heterologous” region of a nucleic acid construct or a vector is a segment of nucleic acid within or attached to another nucleic acid molecule that is not found in association with the other molecule in nature. For example, a heterologous region of a nucleic acid construct could include a coding sequence flanked by sequences not found in association with the coding sequence in nature, i.e., a heterologous promoter. Another example of a heterologous coding sequence is a construct where the coding sequence itself is not found in nature (e.g., synthetic sequences having codons different from the native gene). Similarly, a cell transformed with a construct which is not normally present in the cell would be considered heterologous for purposes of this invention.

By “DNA” is meant a polymeric form of deoxyribonucleotides (adenine, guanine, thymine, or cytosine) in double-stranded or single-stranded form found, inter alia, in linear DNA molecules (e.g., restriction fragments), viruses, plasmids, and chromosomes. In discussing the structure of particular DNA molecules, sequences may be described herein according to the normal convention of giving only the sequence in the 5′ to 3′ direction along the nontranscribed strand of DNA (i.e., the strand having the sequence complementary to the mRNA). The term captures molecules that include the four bases adenine, guanine, thymine, or cytosine, as well as molecules that include base analogues which are known in the art.

As used herein, the terms “complementary” or “complementarity” are used in reference to polynucleotides (i.e., a sequence of nucleotides) related by the base-pairing rules. For example, the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

DNA molecules are said to have “5′ ends” and “3′ ends” because mononucleotides are reacted to make oligonucleotides or polynucleotides in a manner such that the 5′ phosphate of one mononucleotide pentose ring is attached to the 3′ oxygen of its neighbor in one direction via a phosphodiester linkage. Therefore, an end of an oligonucleotide or polynucleotide is referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide or polynucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements that direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

A “gene,” “polynucleotide,” “coding region,” or “sequence” which “encodes” a particular gene product, is a nucleic acid molecule which is transcribed and optionally also translated into a gene product, e.g., a polypeptide, in vitro or in vivo when placed under the control of appropriate regulatory sequences. The coding region may be present in either a cDNA, genomic DNA, or RNA form. When present in a DNA form, the nucleic acid molecule may be single-stranded (i.e., the sense strand) or double-stranded. The boundaries of a coding region are determined by a start codon at the 5′ (amino) terminus and a translation stop codon at the 3′ (carboxy) terminus. A gene can include, but is not limited to, cDNA from prokaryotic or eukaryotic mRNA, genomic DNA sequences from prokaryotic or eukaryotic DNA, and synthetic DNA sequences. Thus, a gene includes a polynucleotide which may include a full-length open reading frame which encodes a gene product (sense orientation) or a portion thereof (sense orientation) which encodes a gene product with substantially the same activity as the gene product encoded by the full-length open reading frame, the complement of the polynucleotide, e.g., the complement of the full-length open reading frame (antisense orientation) and optionally linked 5′ and/or 3′ noncoding sequence(s) or a portion thereof, e.g., an oligonucleotide, which is useful to inhibit transcription, stability or translation of a corresponding mRNA. A transcription termination sequence will usually be located 3′ to the gene sequence.

An “oligonucleotide” includes at least 7 nucleotides, preferably 15, and more preferably 20 or more sequential nucleotides, up to 100 nucleotides, either RNA or DNA, which correspond to the complement of the non-coding strand, or of the coding strand, of a selected mRNA, or which hybridize to the mRNA or DNA encoding the mRNA and remain stably bound under moderately stringent or highly stringent conditions, as defined by methods well known to the art, e.g., in Sambrook et al., A Laboratory Manual, Cold Spring Harbor Press (1989).

The term “control elements” refers collectively to promoter regions, polyadenylation signals, transcription termination sequences, upstream regulatory domains, origins of replication, internal ribosome entry sites (“IRES”), enhancers, splice junctions, and the like, which collectively provide for the replication, transcription, post-transcriptional processing and translation of a coding sequence in a recipient cell. Not all of these control elements need always be present so long as the selected coding sequence is capable of being replicated, transcribed and translated in an appropriate host cell.

The term “promoter region” is used herein in its ordinary sense to refer to a nucleotide region comprising a DNA regulatory sequence, wherein the regulatory sequence is derived from a gene which is capable of binding RNA polymerase and initiating transcription of a downstream (3′ direction) coding sequence. Thus, a “promoter,” refers to a polynucleotide sequence that controls transcription of a gene or coding sequence to which it is operably linked. A large number of promoters, including constitutive, inducible and repressible promoters, from a variety of different sources, are well known in the art.

By “enhancer element” is meant a nucleic acid sequence that, when positioned proximate to a promoter, confers increased transcription activity relative to the transcription activity resulting from the promoter in the absence of the enhancer domain. Hence, an “enhancer” includes a polynucleotide sequence that enhances transcription of a gene or coding sequence to which it is operably linked. A large number of enhancers, from a variety of different sources are well known in the art. A number of polynucleotides which have promoter sequences (such as the commonly-used CMV promoter) also have enhancer sequences.

By “cardiac-specific enhancer or promoter” is meant an element, which, when operably linked to a promoter or alone, respectively, directs gene expression in a cardiac cell and does not direct gene expression in all tissues or all cell types. Cardiac-specific enhancers or promoters may be naturally occurring or non-naturally occurring. One skilled in the art will recognize that the synthesis of non-naturally occurring enhancers or promoters can be performed using standard oligonucleotide synthesis techniques.

“Operably linked” refers to a juxtaposition, wherein the components so described are in a relationship permitting them to function in their intended manner. By “operably linked” with reference to nucleic acid molecules is meant that two or more nucleic acid molecules (e.g., a nucleic acid molecule to be transcribed, a promoter, and an enhancer element) are connected in such a way as to permit transcription of the nucleic acid molecule. A promoter is operably linked to a coding sequence if the promoter controls transcription of the coding sequence. Although an operably linked promoter is generally located upstream of the coding sequence, it is not necessarily contiguous with it. An enhancer is operably linked to a coding sequence if the enhancer increases transcription of the coding sequence. Operably linked enhancers can be located upstream, within or downstream of coding sequences. A polyadenylation sequence is operably linked to a coding sequence if it is located at the downstream end of the coding sequence such that transcription proceeds through the coding sequence into the polyadenylation sequence. “Operably linked” with reference to peptide and/or polypeptide molecules is meant that two or more peptide and/or polypeptide molecules are connected in such a way as to yield a single polypeptide chain, i.e., a fusion polypeptide, having at least one property of each peptide and/or polypeptide component of the fusion. Thus, a signal or targeting peptide sequence is operably linked to another protein if the resulting fusion is secreted from a cell as a result of the presence of a secretory signal peptide or into an organelle as a result of the presence of an organelle targeting peptide.

“Homology” refers to the percent of identity between two polynucleotides or two polypeptides. The correspondence between one sequence and to another can be determined by techniques known in the art. For example, homology can be determined by a direct comparison of the sequence information between two polypeptide molecules by aligning the sequence information and using readily available computer programs. Alternatively, homology can be determined by hybridization of polynucleotides under conditions which form stable duplexes between homologous regions, followed by digestion with single strand-specific nuclease(s), and size determination of the digested fragments. Two DNA, or two polypeptide, sequences are “substantially homologous” to each other when at least about 80%, preferably at least about 90%, and most preferably at least about 95% of the nucleotides, or amino acids, respectively match over a defined length of the molecules, as determined using the methods above.

By “mammal” is meant any member of the class Mammalia including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats, rabbits and guinea pigs, and the like. An “animal” includes vertebrates such as mammals, avians, amphibians, reptiles and aquatic organisms including fish.

By “derived from” is meant that a nucleic acid molecule was either made or designed from a parent nucleic acid molecule, the derivative retaining substantially the same functional features of the parent nucleic acid molecule, e.g., encoding a gene product with substantially the same activity as the gene product encoded by the parent nucleic acid molecule from which it was made or designed.

By “expression construct” or “expression cassette” is meant a nucleic acid molecule that is capable of directing transcription. An expression construct includes, at the least, a promoter. Additional elements, such as an enhancer, and/or a transcription termination signal, may also be included.

The term “exogenous,” when used in relation to a protein, gene, nucleic acid, or polynucleotide in a cell or organism refers to a protein, gene, nucleic acid, or polynucleotide which has been introduced into the cell or organism by artificial or natural means, or in relation a cell refers to a cell which was isolated and subsequently introduced to other cells or to an organism by artificial or natural means. An exogenous nucleic acid may be from a different organism or cell, or it may be one or more additional copies of a nucleic acid which occurs naturally within the organism or cell. An exogenous cell may be from a different organism, or it may be from the same organism. By way of a non-limiting example, an exogenous nucleic acid is in a chromosomal location different from that of natural cells, or is otherwise flanked by a different nucleic acid sequence than that found in nature.

The term “isolated” when used in relation to a nucleic acid, peptide, polypeptide, cell or virus refers to a nucleic acid sequence, peptide, polypeptide, cell or virus that is separated from at least one contaminant nucleic acid, polypeptide, virus or other biological component with which it is ordinarily associated in its natural source. Isolated nucleic acid, peptide, polypeptide, cell or virus are present in a form or setting that is different from that in which it is found in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. The isolated nucleic acid molecule may be present in single-stranded or double-stranded form. When an isolated nucleic acid molecule is to be utilized to express a protein, the molecule will contain at a minimum the sense or coding strand (i.e., the molecule may single-stranded), but may contain both the sense and anti-sense strands (i.e., the molecule may be double-stranded).

The term “recombinant DNA molecule” as used herein refers to a DNA molecule that is comprised of segments of DNA joined together by means of molecular biological techniques.

The term “recombinant protein” or “recombinant polypeptide” as used herein refers to a protein molecule that is expressed from a recombinant DNA molecule.

The term “peptide”, “polypeptide” and protein” are used interchangeably herein unless otherwise distinguished to refer to polymers of amino acids of any length. These terms also include proteins that are post-translationally modified through reactions that include glycosylation, acetylation and phosphorylation.

By “growth factor” is meant an agent that, at least, promotes cell growth or induces phenotypic changes.

### General Overview

Ischemia and reperfusion (I/R)-induced tissue injury are major causes of mortality and morbidity in the civilized world. I/R injury can develop as a consequence of hypotension, shock, or bypass surgery leading to end-organ failure such as acute renal tubular necrosis, liver failure, and bowel infarct. I/R injury can also develop as a result of complications of vascular disease such as stroke, myocardial infarction and solid tumors. For instance, myocardial ischemia, caused by occlusion of coronary artery, is a leading cause for mortality and morbidity worldwide, and results in acute cardiac damage and progressive remodeling that eventually culminate into chronic heart failure. In addition, multiple subclinical I/R incidents can induce cumulative tissue injury leading to chronic degenerative diseases such as vascular dementia, ischemic cardiomyopathy, and renal insufficiency. Cytoprotective strategies using pharmacological agents have yielded limited success in the prevention of I/R injury, e.g., due to the timing of administration of the therapy or achieving adequate tissue levels of therapeutic product.

Moreover, in chronic heart failure, a subnormal level of oxygen is available to the myocardium, e.g., a microischemia that may be global or concentrated in the endocardial layers.

One of the major mechanisms by which cells control gene expression during low oxygen (hypoxia) involves the activation of transcription factor hypoxia-inducible factor 1α (HIF1α), which is quickly degraded during normoxic conditions by ubiquitination mechanisms that include proline hydroxylation and acetylation. Activation of HIF1α leads to transcription of several target genes such as vascular endothelial growth factor (VEGF), erythropoietin, nitric oxide synthase, and several antioxidant enzyme systems such as superoxide dismutase, and heme-oxygenase-1 (HO-1), which may provide protection against I/R injury. However, uncontrolled expression of therapeutic proteins, for instance, vascular endothelial growth factor (VEGF), can cause adverse effects such as hemangioma, retinopathy and occult tumor growth.

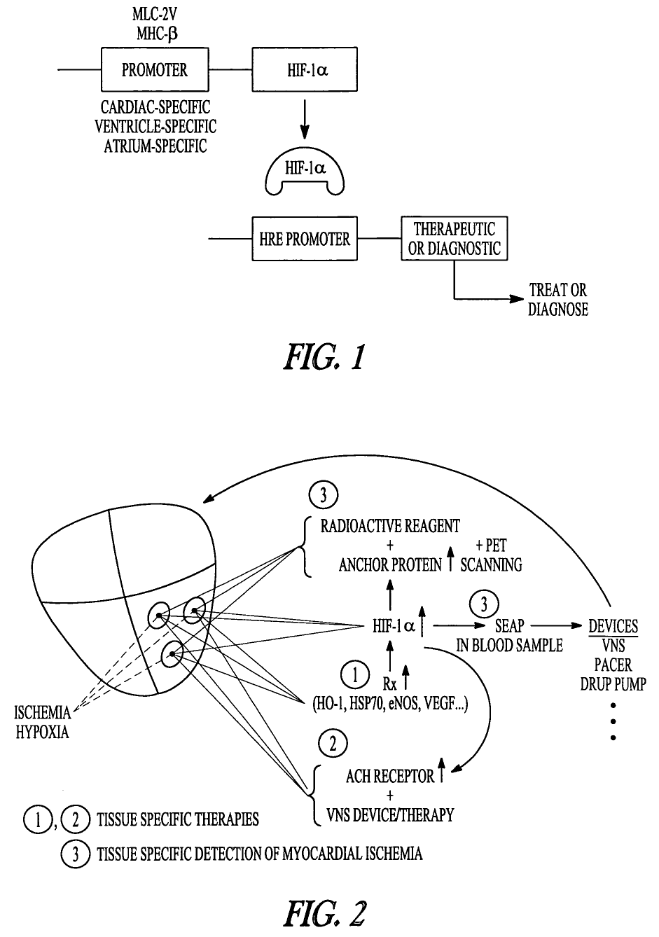

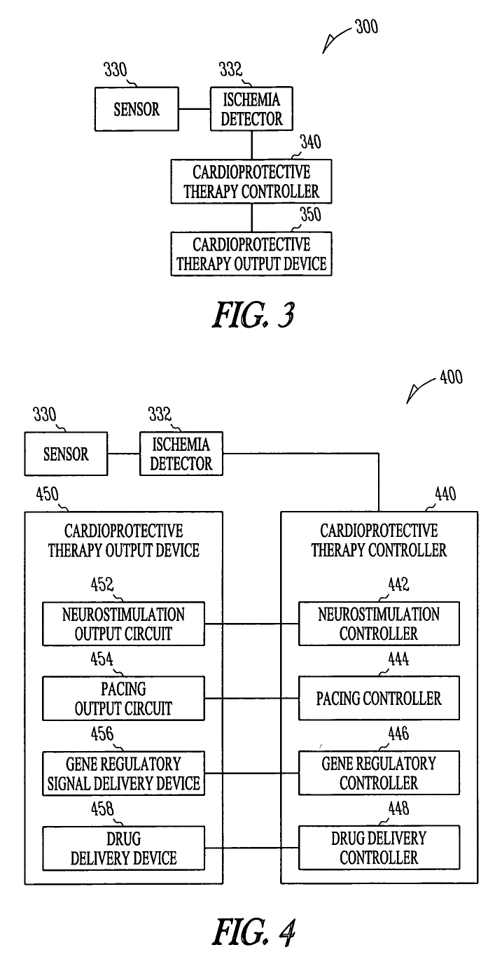

This document describes, among other things, vectors, methods and systems for detecting ischemia, or detecting and delivering one or more therapies for ischemia. In one embodiment, a mammal having or at risk of having ischemia, e.g., cardiac ischemia, is subjected to delivery of a vector system of the invention. One of the vectors of the vector system includes a promoter or enhancer that is organ-, tissue- cell-specific, or energy-regulated, and/or a promoter that is constitutively expressed, operably linked to a hypoxia sensitive gene product. Exemplary energy-regulated transcription elements are described in U.S. patent application Ser. No. 10/788,906, entitled “METHOD AND APPARATUS FOR DEVICE CONTROLLED GENE EXPRESSION”; Ser. No. 11/272,432, entitled “BIOLOGIC DEVICE FOR REGULATION OF GENE EXPRESSION AND METHOD THEREFOR”; Ser. No. 11/276,077, entitled “METHOD AND APPARATUS FOR HEAT OR ELECTROMAGNETIC CONROL OF GENE EXPRESSION”; and Ser. No. 11/424,107, entitled “METHOD TO POSITION THERAPEUTIC AGENTS USING A MAGNETIC FIELD,” all assigned to Cardiac Pacemakers, Inc., which are incorporated by reference herein. In one embodiment, the enhancer may be a muscle creatine kinase (mck) enhancer, and the promoter may be an alpha-myosin heavy chain (MyHC) or beta-MyHC promoter (FIG. 1). In one embodiment, the hypoxia sensitive gene product may be hypoxia-inducible factor 1α (HIF-1α) which accumulates in the cytoplasm and forms functional heterodimers with HIF-1α. In another embodiment, the hypoxia sensitive gene product may be CREB. Another vector encodes at least one therapeutic gene product, cardioprotective gene product or a biomarker, and is operably linked to a transcriptional regulatory region that includes a binding site for the gene product that is stable under hypoxic conditions. In one embodiment, the second vector includes a hypoxia response element (HRE). HRE is a cis-acting element residing in the enhancer of many hypoxia-stimulated genes, including VEGF, erythryopoietin and several glycolytic enzymes. In another embodiment, the vector includes more than one, e.g., tandom, HREs. In one embodiment, the second vector includes a CREB responsive element. In one embodiment, the second vector includes at least one expression cassette that encodes a gene product including, but not limited to, a growth factor, e.g., VEGF, a survival factor, e.g., Akt, a cytokine, a receptor, e.g., acetylcholine receptor (ACHR) or adrenergic receptor, or another gene product, or any combination thereof (FIG. 2). For example, to treat cardiac remodeling associated with heart failure, for instance, dilated cardiomyopathy, the second vector encodes a cardioprotective or therapeutic gene product and under subnormal oxygen levels, the cardioprotective or therapeutic gene product is expressed. The expression of the gene product may inhibit, prevent or reverse cardiac remodeling/dilation, and so be effective for chronic heart failure.

In another embodiment, the second vector encodes a biomarker including a cell associated marker protein such as a fluorescent protein, e.g., green fluorescent protein (GFP), red FP, blue FP or yellow FP, or one which binds a radioactive element or otherwise detectable agent, e.g., binds ¹²⁵I or ¹³¹I, e.g., thyroxine (T4), triiodothyronine (T3), monoiodotyrosine or diiodotyrosine, ¹⁴C, e.g., CmpA which is a cytoplasmic membrane protein involved in HCO₃⁻ uptake, ³²P, e.g., PstS which is involved in inorganic phosphate transport, or Gd or radioactive isotopes thereof, e.g., ¹⁵²Gd. In one embodiment, under normoxic conditions, there is a basal level or no expression of the therapeutic or cardioprotective gene product, or biomarker.

Optionally, more than two vectors may be employed, each with a different open reading frame linked to an organ-, tissue-, cell-specific, or energy-regulated, transcriptional regulatory element, or a transcriptional regulatory region that includes one or more binding sites for a gene product that is unstable under normoxic conditions and stable under hypoxic conditions. For instance, one vector has an organ-, tissue-, cell-specific, or energy-regulated, transcriptional control element linked to the gene product that is unstable under normoxic conditions and stable under hypoxic condition, and two or more vectors have a transcriptional regulatory region that includes one or more binding sites for the gene product, where each of those transcriptional regulatory regions is linked to an open reading frame for different gene products.

The vectors may be administered to a mammal by any route or in any delivery vehicle. For instance, a plasmid may contain both vectors and may be injected into regions of the heart. In another embodiment, replication incompetent viral vectors may be locally administered to one or more physiological sites in a mammal. In yet another embodiment, the vectors are delivered to cells ex vivo, and those recombinant cells may be administered to a mammal, e.g., to one or more cardiac locations.

In one embodiment, prior to, concurrent with or after vector delivery to a mammal, an implantable device for electrical or drug therapy is provided to the mammal to enhance the efficacy of the vector system. In one embodiment, the device is introduced at or near damaged cardiovascular tissue. In response to detection of ischemia, the device emits electrical stimulation or a drug. In one embodiment, after a desirable change in ischemia is detected, the electrical or drug therapy is discontinued. In another embodiment, the electrical or drug therapy is delivered for a predetermined time period.

Thus, this document discusses a vector system that includes at least two expression cassettes. In response to hypoxic conditions, the vector system expresses a biomarker, or a therapeutic or cardioprotective gene product. In a further embodiment, the detection and gene therapy is performed in conjunction with electrical therapy, such as pacing therapy, and/or drug therapy. One specific example of the implantable medical device is an implantable cardiac rhythm management (CRM) device.

### Gene Delivery Vectors

Gene delivery vectors include, for example, viral vectors, liposomes and other lipid-containing complexes, and other macromolecular complexes capable of mediating delivery of a gene to a host cell. Vectors can also comprise other components or functionalities that further modulate gene delivery and/or gene expression, or that otherwise provide beneficial properties to the targeted cells. Such other components include, for example, components that influence binding or targeting to cells (including components that mediate cell-type or tissue-specific binding); components that influence uptake of the vector by the cell; components that influence localization of the transferred gene within the cell after uptake (such as agents mediating nuclear localization); and components that influence expression of the gene. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. Selectable markers can be positive, negative or bifunctional. Positive selectable markers allow selection for cells carrying the marker, whereas negative selectable markers allow cells carrying the marker to be selectively eliminated. A variety of such marker genes have been described, including bifunctional (i.e., positive/negative) markers (see, e.g., WO 92/08796; and WO 94/28143). Such marker genes can provide an added measure of control that can be advantageous in gene therapy contexts. A large variety of such vectors are known in the art and are generally available.

Gene delivery vectors within the scope of the invention include, but are not limited to, isolated nucleic acid, e.g., plasmid-based vectors which may be extrachromosomally maintained, and viral vectors, e.g., recombinant adenovirus, retrovirus, lentivirus, herpesvirus, poxvirus, papilloma virus, or adeno-associated virus, including viral and non-viral vectors which are present in liposomes, e.g., neutral or cationic liposomes, such as DOSPA/DOPE, DOGS/DOPE or DMRIE/DOPE liposomes, and/or associated with other molecules such as DNA-anti-DNA antibody-cationic lipid (DOTMA/DOPE) complexes. Exemplary gene delivery vectors are described below. Gene delivery vectors may be administered via any route including, but not limited to, intramuscular, buccal, rectal, intravenous or intracoronary administration, and transfer to cells may be enhanced using electroporation and/or iontophoresis, and/or scaffolding such as extracellular matrix or hydrogels, e.g., a hydrogel patch.

### Retroviral Vectors

Retroviral vectors exhibit several distinctive features including their ability to stably and precisely integrate into the host genome providing long-term transgene expression. These vectors can be manipulated ex vivo to eliminate infectious gene particles to minimize the risk of systemic infection and patient-to-patient transmission. Pseudotyped retroviral vectors can alter host cell tropism.

### Lentiviruses

Lentiviruses are derived from a family of retroviruses that include human immunodeficiency virus and feline immunodeficiency virus. However, unlike retroviruses that only infect dividing cells, lentiviruses can infect both dividing and nondividing cells. For instance, lentiviral vectors based on human immunodeficiency virus genome are capable of efficient transduction of cardiac myocytes in vivo. Although lentiviruses have specific tropisms, pseudotyping the viral envelope with vesicular stomatitis virus yields virus with a broader range (Schnepp et al., Meth. Mol. Med., 69:427 (2002)).

### Adenoviral Vectors

Adenoviral vectors may be rendered replication-incompetent by deleting the early (E1A and E1B) genes responsible for viral gene expression from the genome and are stably maintained into the host cells in an extrachromosomal form. These vectors have the ability to transfect both replicating and nonreplicating cells and, in particular, these vectors have been shown to efficiently infect cardiac myocytes in vivo, e.g., after direction injection or perfusion. Adenoviral vectors have been shown to result in transient expression of therapeutic genes in vivo, peaking at 7 days and lasting approximately 4 weeks. The duration of transgene expression may be improved in systems utilizing cardiac specific promoters. In addition, adenoviral vectors can be produced at very high titers, allowing efficient gene transfer with small volumes of virus.

### Adeno-Associated Virus Vectors

Recombinant adeno-associated viruses (rAAV) are derived from nonpathogenic parvoviruses, evoke essentially no cellular immune response, and produce transgene expression lasting months in most systems. Moreover, like adenovirus, adeno-associated virus vectors also have the capability to infect replicating and nonreplicating cells and are believed to be nonpathogenic to humans. Moreover, they appear promising for sustained cardiac gene transfer (Hoshijima et al,. Nat. Med., 8:864 (2002); Lynch et al., Circ. Res., 80:197 (1997)).

### Herpesvirus/Amplicon

Herpes simplex virus 1 (HSV-1) has a number of important characteristics that make it an important gene delivery vector in vivo. There are two types of HSV-1-based vectors: 1) those produced by inserting the exogenous genes into a backbone virus genome, and 2) HSV amplicon virions that are produced by inserting the exogenous gene into an amplicon plasmid that is subsequently replicated and then packaged into virion particles. HSV-1 can infect a wide variety of cells, both dividing and nondividing, but has obviously strong tropism towards nerve cells. It has a very large genome size and can accommodate very large transgenes (>35 kb). Herpesvirus vectors are particularly useful for delivery of large genes, e.g., genes encoding ryanodine receptors and titin.

### Plasmid DNA Vectors

Plasmid DNA is often referred to as “naked DNA” to indicate the absence of a more elaborate packaging system. Direct injection of plasmid DNA to myocardial cells in vivo has been accomplished. Plasmid-based vectors are relatively nonimmunogenic and nonpathogenic, with the potential to stably integrate in the cellular genome, resulting in long-term gene expression in postmitotic cells in vivo. For example, expression of secreted angiogenesis factors after muscle injection of plasmid DNA, despite relatively low levels of focal transgene expression, has demonstrated significant biologic effects in animal models and appears promising clinically (Isner, Nature, 415:234 (2002)). Furthermore, plasmid DNA is rapidly degraded in the blood stream; therefore, the chance of transgene expression in distant organ systems is negligible. Plasmid DNA may be delivered to cells as part of a macromolecular complex, e.g., a liposome or DNA-protein complex, and delivery may be enhanced using techniques including electroporation.

### Transcriptional Control Elements

In some embodiments, organ-, cell- or tissue-specific control elements, such as muscle-specific and inducible promoters, enhancers and the like, will be of particular use. Such control elements include, but are not limited to, those derived from the actin and myosin gene families, such as from the myoD gene family (Weintraub et al., Science, 251, 761 (1991)); the myocyte-specific enhancer binding factor MEF-2 (Cseijesi and Olson, Mol. Cell Biol., 11, 4854 (1991)); control elements derived from the human skeletal actin gene (Muscat et al., Mol. Cell Bio., 7, 4089 (1987)) and the cardiac actin gene; muscle creatine kinase sequence elements (Johnson et al., Mol. Cell Biol., 9, 3393 (1989)) and the murine creatine kinase enhancer (mCK) element; control elements derived from the skeletal fast-twitch troponin C gene, the slow-twitch cardiac troponin C gene and the slow-twitch troponin I genes.

Cardiac cell restricted promoters include but are not limited to promoters from the following genes: a α-myosin heavy chain gene, e.g., a ventricular α-myosin heavy chain gene, β-myosin heavy chain gene, e.g., a ventricular β-myosin heavy chain gene, myosin light chain 2v gene, e.g., a ventricular myosin light chain 2 gene, myosin light chain 2a gene, e.g., a ventricular myosin light chain 2 gene, cardioyocyte-restricted cardiac ankyrin repeat protein (CARP) gene, cardiac α-actin gene, cardiac m2 muscarinic acetylcholine gene, ANP gene, BNP gene, cardiac troponin C gene, cardiac troponin I gene, cardiac troponin T gene, cardiac sarcoplasmic reticulum Ca-ATPase gene, skeletal a-actin gene, as well as an artificial cardiac cell-specific promoter.

Further, chamber-specific promoters or enhancers may also be employed, e.g., for atrial-specific expression, the quail slow myosin chain type 3 (MyHC3) or ANP promoter, or the cGATA-6 enhancer, may be employed. For ventricle-specific expression, the iroquois homeobox gene may be employed. Examples of ventricular myocyte-specific promoters include a ventricular myosin light chain 2 promoter and a ventricular myosin heavy chain promoter.

In other embodiments, disease-specific control elements may be employed, e.g., hypoxia-specific control element. Thus, control elements from genes associated with a particular disease, including but not limited to any of the genes disclosed herein may be employed in vectors of the invention.

Nevertheless, other promoters and/or enhancers which are not specific for cardiac cells or muscle cells, e.g., RSV promoter, may be employed in the expression cassettes and methods of the invention. Other sources for promoters and/or enhancers are promoters and enhancers from the Csx/NKX 2.5 gene, titin gene, α-actinin gene, myomesin gene, M protein gene, cardiac troponin T gene, RyR2 gene, Cx40 gene, and Cx43 gene, as well as genes which bind Mef2, dHAND, GATA, CarG, E-box, Csx/NKX 2.5, or TGF-beta, or a combination thereof.

### Targeted Vectors

The present invention contemplates the use of targeted vector constructs having features that tend to target gene delivery and/or gene expression to particular host cells or host cell types (such as the myocardium). Such targeted vector constructs would thus include targeted delivery vectors and/or targeted vectors, as described herein. Restricting delivery and/or expression can be beneficial as a means of further focusing the potential effects of gene delivery. The potential usefulness of further restricting delivery/expression depends in large part on the type of vector being used and the method and place of introduction of such vector. For instance, delivery of viral vectors via intracoronary injection to the myocardium has been observed to provide, in itself, highly targeted gene delivery. In addition, using vectors that do not result in transgene integration into a replicon of the host cell (such as adenovirus and numerous other vectors), cardiac myocytes are expected to exhibit relatively long transgene expression since the cells do not undergo rapid turnover. In contrast, expression in more rapidly dividing cells would tend to be decreased by cell division and turnover. However, other means of limiting delivery and/or expression can also be employed, in addition to or in place of the illustrated delivery method, as described herein.

Targeted delivery vectors include, for example, vectors (such as viruses, non-viral protein-based vectors and lipid-based vectors) having surface components (such as a member of a ligand-receptor pair, the other half of which is found on a host cell to be targeted) or other features that mediate preferential binding and/or gene delivery to particular host cells or host cell types. As is known in the art, a number of vectors of both viral and non-viral origin have inherent properties facilitating such preferential binding and/or have been modified to effect preferential targeting (see, e.g., Miller, et al., FASEB Journal, 9:190 (1995); Chonn et al., Curr. Opin. Biotech., 6:698 (1995); Schofield et al., British Med. Bull., 51:56 (1995); Schreier, Pharmaceutica Acta Helvetiae, 68:145 (1994); Ledley, Human Gene Therapy, 6:1129 (1995); WO 95/34647; WO 95/28494; and WO 96/00295).

Targeted vectors include vectors (such as viruses, non-viral protein-based vectors and lipid-based vectors) in which delivery results in transgene expression that is relatively limited to particular host cells or host cell types. For example, transgenes can be operably linked to heterologous tissue-specific enhancers or promoters thereby restricting expression to cells in that particular tissue. For example, tissue-specific transcriptional control sequences derived from a gene encoding left ventricular myosin light chain-2 (MLC₂V) or myosin heavy chain (MHC) can be fused to a transgene within a vector. Expression of the transgene can therefore be relatively restricted to ventricular cardiac myocytes.

### Sources of Donor Cells

Sources for donor cells to deliver the vector system of the invention include but are not limited to bone marrow-derived cells, e.g., mesenchymal cells and stromal cells, smooth muscle cells, fibroblasts, SP cells, pluripotent cells or totipotent cells, e.g., teratoma cells, hematopoietic stem cells, for instance, cells from cord blood and isolated CD34⁺ cells, multipotent adult progenitor cells, adult stem cells, embyronic stem cells, skeletal muscle derived cells, for instance, skeletal muscle cells and skeletal myoblasts, cardiac derived cells, myocytes, e.g., ventricular myocytes, atrial myocytes, SA nodal myocytes, AV nodal myocytes, and Purkinje cells. In one embodiment, the donor cells are autologous cells, however, non-autologous cells, e.g., xenogeneic cells, may be employed. The donor cells can be expanded in vitro to provide an expanded population of donor cells for administration to a recipient mammal. In addition, donor cells may be treated in vitro as exemplified below. Sources of donor cells and methods of culturing those cells are known to the art.

Donor cells may also be treated in vitro by subjecting them to mechanical, electrical, or biological conditioning, or any combination thereof, as described in U.S. patent application Ser. No. 10/722,115, entitled “METHOD AND APPARATUS FOR CELL AND ELECTRICAL THERAPY OF LIVING TISSUE”, which is incorporated by reference herein, conditioning which may include continuous or intermittent exposure to the exogenous stimuli. For instance, biological conditioning includes subjecting donor cells to exogenous agents, e.g., differentiation factors, growth factors, angiogenic proteins, survival factors, and cytokines. Preferred exogenous agents include those which enhance the localization, engraftment, differentiation, proliferation and/or function of donor cells after transplant. In one embodiment, the genetically modified (transgenic) donor cells, besides having the vector system of the invention, may include an expression cassette, the expression of which in donor cells enhances proliferation, localization, engraftment, differentiation and/or function of the donor cells after implantation.

### Routes of Administration, Dosages and Dosage Forms

Administration of the gene delivery vectors in accordance with the present invention may be continuous or intermittent, depending, for example, upon the recipient's physiological condition, whether the purpose of the administration is therapeutic or prophylactic, and other factors known to skilled practitioners. The administration of the gene delivery vectors may be essentially continuous over a preselected period of time or may be in a series of spaced doses. Both local and systemic administration is contemplated.

One or more suitable unit dosage forms comprising the gene delivery vectors, which may optionally be formulated for sustained release, can be administered by a variety of routes including oral, or parenteral, including by rectal, buccal, vaginal and sublingual, transdermal, subcutaneous, intravenous, intramuscular, intraperitoneal, intrathoracic, intrapulmonary and intranasal routes. The formulations may, where appropriate, be conveniently presented in discrete unit dosage forms and may be prepared by any of the methods well known to pharmacy. Such methods may include the step of bringing into association the vector with liquid carriers, solid matrices, semi-solid carriers, finely divided solid carriers or combinations thereof, and then, if necessary, introducing or shaping the product into the desired delivery system.

The amount of gene delivery vector(s), e.g., those which are present in a recombinant cell or in acellular form, administered to achieve a particular outcome will vary depending on various factors including, but not limited to, the genes and promoters chosen, the condition, patient specific parameters, e.g., height, weight and age, and whether diagnosis, or prevention or treatment, is to be achieved. The gene delivery vector system of the invention is amenable to chronic use for prophylactic purposes.

Vectors of the invention may conveniently be provided in the form of formulations suitable for administration, e.g., into the blood stream (e.g., in an intracoronary artery). A suitable administration format may best be determined by a medical practitioner for each patient individually, according to standard procedures. Suitable pharmaceutically acceptable carriers and their formulation are described in standard formulations treatises, e.g., Remington's Pharmaceuticals Sciences. Vectors of the present invention should preferably be formulated in solution at neutral pH, for example, about pH 6.5 to about pH 8.5, more preferably from about pH 7 to 8, with an excipient to bring the solution to about isotonicity, for example, 4.5% mannitol or 0.9% sodium chloride, pH buffered with art-known buffer solutions, such as sodium phosphate, that are generally regarded as safe, together with an accepted preservative such as metacresol 0.1% to 0.75%, more preferably from 0.15% to 0.4% metacresol. Obtaining a desired isotonicity can be accomplished using sodium chloride or other pharmaceutically acceptable agents such as dextrose, boric acid, sodium tartrate, propylene glycol, polyols (such as mannitol and sorbitol), or other inorganic or organic solutes. Sodium chloride is preferred particularly for buffers containing sodium ions. If desired, solutions of the above compositions can also be prepared to enhance shelf life and stability. Therapeutically useful compositions of the invention can be prepared by mixing the ingredients following generally accepted procedures. For example, the selected components can be mixed to produce a concentrated mixture which may then be adjusted to the final concentration and viscosity by the addition of water and/or a buffer to control pH or an additional solute to control tonicity.

The vectors can be provided in a dosage form containing an amount of a vector effective in one or multiple doses. For viral vectors, the effective dose may be in the range of at least about 10⁷ viral particles, preferably about 10⁹ viral particles, and more preferably about 10¹¹ viral particles. The number of viral particles may, but preferably does not exceed 10¹⁴. As noted, the exact dose to be administered is determined by the attending clinician, but is preferably in 1 ml phosphate buffered saline. For delivery of recombinant cells, the number of cells to be administered will be an amount which results in a beneficial effect to the recipient. For example, from 10² to 10¹⁰, e.g., from 10³ to 10⁹, 10⁴ to 10⁸, or 10⁵ to 10⁷, cells can be administered. For delivery of plasmid DNA alone, or plasmid DNA in a complex with other macromolecules, the amount of DNA to be administered will be an amount which results in a beneficial effect to the recipient. For example, from 0.0001 to 1 mg or more, e.g., up to 1 g, in individual or divided doses, e.g., from 0.001 to 0.5 mg, or 0.01 to 0.1 mg, of DNA can be administered.

In one embodiment, in the case of heart disease, administration may be by intracoronary injection to one or both coronary arteries (or to one or more saphenous vein or internal mammary artery grafts or other conduits) using an appropriate coronary catheter. A variety of catheters and delivery routes can be used to achieve intracoronary delivery, as is known in the art. For example, a variety of general purpose catheters, as well as modified catheters, suitable for use in the present invention are available from commercial suppliers. Also, where delivery to the myocardium is achieved by injection directly into a coronary artery, a number of approaches can be used to introduce a catheter into the coronary artery, as is known in the art. By way of illustration, a catheter can be conveniently introduced into a femoral artery and threaded retrograde through the iliac artery and abdominal aorta and into a coronary artery. Alternatively, a catheter can be first introduced into a brachial or carotid artery and threaded retrograde to a coronary artery. Detailed descriptions of these and other techniques can be found in the art (see, e.g., above, including: Topol, (ed.), The Textbook of Interventional Cardiology, 4th Ed. (Elsevier 2002); Rutherford, Vascular Surgery, 5th Ed. (W. B. Saunders Co. 2000); Wyngaarden et al. (eds.), The Cecil Textbook of Medicine, 22nd Ed. (W. B. Saunders, 2001); and Sabiston, The Textbook of Surgery, 16th Ed. (Elsevier 2000)).

By way of illustration, liposomes and other lipid-containing gene delivery complexes can be used to deliver one or more transgenes. The principles of the preparation and use of such complexes for gene delivery have been described in the art (see, e.g., Ledley, Human Gene Therapy, 6:1129 (1995); Miller et al., FASEB Journal, 9:190 (1995); Chonn et al., Curr. Opin. Biotech., 6:698 (1995); Schofield et al., British Med. Bull., 51:56 (1995); Brigham et al., J. Liposome Res., 3:31 (1993)).

Pharmaceutical formulations containing the gene delivery vectors can be prepared by procedures known in the art using well known and readily available ingredients. For example, the agent can be formulated with common excipients, diluents, or carriers, and formed into tablets, capsules, suspensions, powders, and the like. The vectors of the invention can also be formulated as elixirs or solutions for convenient oral administration or as solutions appropriate for parenteral administration, for instance by intramuscular, subcutaneous or intravenous routes.

The pharmaceutical formulations of the vectors can also take the form of an aqueous or anhydrous solution or dispersion, or alternatively the form of an emulsion or suspension.

In one embodiment, the vectors may be formulated for parenteral administration (e.g., by injection, for example, bolus injection or continuous infusion) and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

These formulations can contain pharmaceutically acceptable vehicles and adjuvants which are well known in the prior art. It is possible, for example, to prepare solutions using one or more organic solvent(s) that is/are acceptable from the physiological standpoint.

For administration to the upper (nasal) or lower respiratory tract by inhalation, the vector is conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Alternatively, for administration by inhalation or insufflation, the composition may take the form of a dry powder, for example, a powder mix of the therapeutic agent and a suitable powder base such as lactose or starch. The powder composition may be presented in unit dosage form in, for example, capsules or cartridges, or, e.g., gelatine or blister packs from which the powder may be administered with the aid of an inhalator, insufflator or a metered-dose inhaler.

For intra-nasal administration, the vector may be administered via nose drops, a liquid spray, such as via a plastic bottle atomizer or metered-dose inhaler. Typical of atomizers are the Mistometer (Wintrop) and the Medihaler (Riker).

The local delivery of the vectors can also be by a variety of techniques which administer the vector at or near the site of disease. Examples of site-specific or targeted local delivery techniques are not intended to be limiting but to be illustrative of the techniques available. Examples include local delivery catheters, such as an infusion or indwelling catheter, e.g., a needle infusion catheter, shunts and stents or other implantable devices, site specific carriers, direct injection, or direct applications.

For topical administration, the vectors may be formulated as is known in the art for direct application to a target area. Conventional forms for this purpose include wound dressings, coated bandages or other polymer coverings, ointments, creams, lotions, pastes, jellies, sprays, and aerosols, as well as in toothpaste and mouthwash, or by other suitable forms. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents. The active ingredients can also be delivered via iontophoresis, e.g., as disclosed in U.S. Pat. Nos. 4,140,122; 4,383,529; or 4,051,842. The percent by weight of a therapeutic agent of the invention present in a topical formulation will depend on various factors, but generally will be from 0.01% to 95% of the total weight of the formulation, and typically 0.1-25% by weight.

When desired, the above-described formulations can be adapted to give sustained release of the active ingredient employed, e.g., by combination with certain hydrophilic polymer matrices, e.g., comprising natural gels, synthetic polymer gels or mixtures thereof.

Drops, such as eye drops or nose drops, may be formulated with an aqueous or non-aqueous base also comprising one or more dispersing agents, solubilizing agents or suspending agents. Liquid sprays are conveniently delivered from pressurized packs. Drops can be delivered via a simple eye dropper-capped bottle, or via a plastic bottle adapted to deliver liquid contents dropwise, via a specially shaped closure.

The vector may further be formulated for topical administration in the mouth or throat. For example, the active ingredients may be formulated as a lozenge further comprising a flavored base, usually sucrose and acacia or tragacanth; pastilles comprising the composition in an inert base such as gelatin and glycerin or sucrose and acacia; mouthwashes comprising the composition of the present invention in a suitable liquid carrier; and pastes and gels, e.g., toothpastes or gels, comprising the composition of the invention.

The formulations and compositions described herein may also contain other ingredients such as antimicrobial agents or preservatives.

### Exemplary Routes for Cardiac Delivery

Several techniques have been developed for cardiac gene delivery, including pericardial infusion, endomyocardial injection, intracoronary injection, coronary venous retroperfusion, and aortic root injection (Isner, Nature, 415:234 (2002)). The different techniques achieve variable response in homogeneity of gene delivery, resulting in focal gene expression within the heart (Hajjar et al., Circ. Res., 86:616 (2000). For this reason, techniques that achieve diffuse uptake would seem to be superior. Two such methods utilize the heart's arterial and venous circulation to accomplish disseminated viral transfection. Arterial injection, performed directly through a percutaneous approach or indirectly by an infusion into the cross-clamped aorta, has shown promise in animal models of heart failure and is appealing in that it can be performed either at the time of cardiac surgery or as percutaneous intervention (Hajjar et al., PNAS USA, 95:5251 (1998)). Similarly, retroperfusion through the coronary sinus appears to produce a more global gene expression in comparison with techniques of localized or focal injection (Boeckstegers et al., Circ., 100:1 (1999)).

Recombinant cells may be administered intravenously, transvenously, intramyocardially or by any other convenient route, and delivered by a needle, catheter, e.g., a catheter which includes an injection needle or infusion port, or other suitable device.

### Direct Myocardial Injection

Direct myocardial injection of plasmid DNA as well as virus vectors, e.g., adenoviral vectors, and cells including recombinant cells has been documented in a number of in vivo studies. This technique when employed with plasmid DNA or adenoviral vectors has been shown to result in effective transduction of cardiac myocytes. Thus, direct injection may be employed as an adjunct therapy in patients undergoing open-heart surgery or as a stand-alone procedure via a modified thorascope through a small incision. In one embodiment, this mode of administration is used to deliver a gene or gene product that would only require limited transfection efficiency to produce a significant therapeutic response, such as a gene that encodes for or leads to a secreted product (e.g., VEGF, endothelial nitric oxide synthase). Virus, e.g., pseudotyped, or DNA- or virus-liposome complexes may be delivered intramyocardially.

### Catheter-Based Delivery

Intracoronary delivery of genetic material can result in transduction of approximately 30% of the myocytes predominantly in the distribution of the coronary artery. Parameters influencing the delivery of vectors via intracoronary perfusion and enhancing the proportion of myocardium transduced include a high coronary flow rate, longer exposure time, vector concentration, and temperature. Gene delivery to a substantially greater percent of the myocardium may be enhanced by administering the gene in a low-calcium, high-serotonin mixture (Donahue et al., Nat. Med., 6:1395 (2000)). The potential use of this approach for gene therapy for heart failure may be increased by the use of specific proteins that enhance myocardial uptake of vectors (e.g., cardiac troponin T).

Improved methods of catheter-based gene delivery have been able to achieve almost complete transfection of the myocardium in vivo. Hajjar et al. (Proc. Natl. Acad. Sci. USA, 95:5251 (1998)) used a technique combining surgical catheter insertion through the left ventricular apex and across the aortic valve with perfusion of the gene of interest during cross-clamping of the aorta and pulmonary artery. This technique resulted in almost complete transduction of the heart and could serve as a protocol for the delivery of adjunctive gene therapy during open-heart surgery when the aorta can be cross-clamped.

Recombinant cells may also be delivered via catheter.

### Pericardial Delivery

Gene delivery to the ventricular myocardium by injection of genetic material into the pericardium has shown efficient gene delivery to the epicardial layers of the myocardium. However, hyaluronidase and collagenase may enhance transduction without any detrimental effects on ventricular function. Recombinant cells may also be delivered pericardially.

### Intravenous Delivery

Intravenous gene delivery may be efficacious for myocardial gene delivery.

However, to improve targeted delivery and transduction efficiency of intravenously administered vectors, targeted vectors may be employed. In one embodiment, intravenous administration of DNA-liposome or antibody-DNA complexes may be employed.

### Lead-Based Delivery

Gene delivery can be performed by incorporating a gene delivery device or lumen into a lead such as a pacing lead, defibrillation lead, or pacing-defibrillation lead. An endocardial lead including a gene delivery device or lumen allows gene delivery to the endocardial layers of the myocardium. An epicardial lead including a gene delivery device or lumen allows gene delivery to the endocardial layers of the myocardium. A transvenous lead including a gene delivery device or lumen may also allow intravenous gene delivery. Lead-based delivery is particularly advantageous when the lead is used to deliver electrical and gene therapies to the same region.

Generally any route of administration may be employed, including oral, mucosal, intramuscular, buccal and rectal administration. Fro certain vectors, certain route of administration may be preferred. For instance, viruses, e.g., pseudotypsed virus, and DNA- or virus-liposome, e.g., HVJ-liposome, may be administered by coronary infusion, while HVJ-liposome complexes may be delivered pericardially.

Recombinant cells may also be delivered systemically, e.g., intravenously.

### Exemplary Vector Systems

As discussed herein, the system provides for automatic detection of ischemia, or automatic detection and treatment that can either rescue or protect the tissue at risk of ischemia. The system is based on hypoxia sensitive gene products, e.g., hypoxia-inducible factor (HIF), e.g., see NCBI Accession Nos. NP001521, NP0851397, Q16665, AAC50152, CAH17551, or AAC68568, or CREB, e.g., see NCBI Accession Nos. AAD13869, P16220, P15337, AAL47131, P27925, NP604391 and NP004370, the disclosures of which are incorporated by reference herein and provides detection specific or therapy or ischemia at one or more locations after ischemic events. In nornoxic condition, a hypoxia sensitive gene product such as HIF-1α protein is not stable and degrades quickly. In one embodiment, the hypoxia sensitive gene is downstream of a cardiac-specific promoter, e.g., one having MLC-2v or MHC-β. Under the hypoxic conditions, the hypoxia sensitive gene product is stable and interacts with a HRE containing transcription regulation region, and activates the expression of therapeutic or biomarker. Thus, the system provides a unique spatial specificity for the treatment or cardioprotection, for instance, of the heart, that is activated promptly and automatically by expression of a hypoxia sensitive gene product and ischemia-inducible expression of genes beneficial to the heart, which are linked to HREs. The system is a closed-loop system that includes at least one vector set that senses hypoxia (ischemia) and transactivates a gene of interest, e.g., a therapeutic or reporter gene of interest, and optionally a device that delivers electrical or drug therapy. In one embodiment, the beneficial gene encodes a substrate for device therapy. For example, the beneficial gene may be one encoding AChR (e.g., muscarinic receptor). An increase in expression of AChR receptor at ischemic site(s) in the heart, which is induced by the sustained expression of a hypoxia stable gene product, may provide an elevated substrate (receptor) for vagal stimulation therapy (VST) to work more effectively.

Other therapeutic or cardioprotective gene of interest include but are not limited to those endothelial nitric oxide synthase (eNOS), e.g., see NCBI Accession Nos. NP000594, CAA53950, AAK71989, or BAA05652, the disclosures of which are incorporated by reference herein; hemeoxygenase, e.g., HO-1, e.g., see NCBI Accession Nos. CAA32886, P09601, or P14901, the disclosures of which are incorporated by reference herein; heat shock proteins (HSPs), for instance, HSP70, e.g., see NCBI Accession Nos. 156208, NP005336, NP694881, AAK17898, NP005337 or AAA02807, the disclosures of which are incorporated by reference herein; a cytokine, a survival factor, e.g., Akt, e.g., see NCBI Accession Nos. NP001014432, NP001014431 or NP005154, the disclosures of which are incorporated by reference herein; or a growth factor such as VEGF, e.g., see NCBI Accession Nos. AAK95844, AAC63143, CAI19965, CAC19516, CAC19515, CAC19514, CAC19513, CAC19512, AAV34601, or AAL27630, the disclosures of which are incorporated by reference herein, that likely are therapeutic and cardioprotective if promptly turned on at the location of myocardial ischemia.

The system also allows ischemia-inducible expression of biomarkers that provide specific location information of organs or tissues at risk as guide for effective therapies. One example of a system for myocardial ischemia includes a reporter gene that encodes a protein that binds or attracts radioactive reagent, e.g., a human sodium iodide symporter (hNIS) which binds radioactive sodium pertechnetate Na⁹⁹mTcO₄ for a certain period of time, a gene product which binds radioactive phosphorus, carbon, iodide, or other molecules useful in diagnostics, e.g., gadolinium which may be employed for magnetic resonance imaging. In one embodiment, the expression of a protein that binds sodium or an analog thereof, e.g., Group IA metals, at the site of ischemia provides a location marker for detection of myocardial ischemia by non-invasive PET scanning or other radiation-based equipment.

As another example, the gene of interest could be a reporter gene encoding a secreted protein such as secreted alkaline phosphatase (SEAP). SEAP may conveniently be collected from a blood sample and an elevation of SEAP level induced by HIF-1α expression may indicate a myocardial ischemia or a heart at risk of MI. Such a signal can be used as an indication for appropriate device intervention, e.g., a pacer or a drug pump.

### Devices

The gene-based ischemia detection and treatment discussed in this document may be combined with a device therapy. The device therapy includes one or more cardioprotective therapies delivered by a device such as an implantable medical device. In one embodiment, a cardioprotective device is used to enhance the effectiveness of a gene therapy. In another embodiment, a cardioprotective device delivers one or more cardioprotective therapies in response to a detection of ischemia using a biologic detection method discussed in this document.

FIG. 3 is a block diagram showing an embodiment of a cardioprotective device 300 for delivering the device therapy. Device 300 includes a sensor 330, an ischemia detector 332, a cardioprotective therapy controller 340, and a cardioprotective therapy output device 350.

Sensor 330 senses one or more physiological parameters, or changes in the one or more physiological parameters, that indicate of an ischemic event. Examples of the ischemic event include an acute myocardial infarction and a detectable condition indicative of a substantial risk of myocardial infarction. Ischemia detector 332 detects the ischemic event from the one or more physiological parameters, or the changes in the one or more physiological parameters. In response to the detection of the ischemic event, cardioprotective therapy controller 340 initiates and controls the delivery of one or more cardioprotective therapies. Cardioprotective therapy output device 350 delivers the one or more cardioprotective therapies.

Ischemia detector 332 includes an ischemia analyzer running an automatic ischemia detection algorithm to detect the ischemic event from the one or more physiological parameters or changes therein. In one embodiment, ischemia detector 332 produces an ischemia alert signal indicative of the detection of each ischemic event. In one embodiment, in response to the ischemia alert signal, device 300 produces an alarm signal, such as a predetermined audio tone, that is perceivable by the patient. In another embodiment, the ischemia alert signal is transmitted to a remote location for producing an alarm signal and/or a warning message for a physician or other caregiver.

In one embodiment, ischemia detector 332 detects the ischemic events from a parameter indicative of a SEAP level. Sensor 330 senses SEAP in blood and produces the parameter indicative of the SEAP level. Ischemia detector 332 detects the ischemic event by comparing the parameter indicative of the SEAP level to a threshold. The ischemic event is detected when the parameter indicative of the SEAP level exceeds the threshold, i.e., when the blood SEAP level has elevated to a certain level.

In another embodiment, ischemia detector 332 detects the ischemic events from one or more cardiac parameters or changes therein. Sensor 330 includes a cardiac sensing circuit. In a specific embodiment, sensor 330 includes an electrogram sensing circuit that senses one or more electrograms, and ischemia detector 332 detects the ischemic events from the one or more electrograms. Examples of an electrogram-based ischemia detector are discussed in U.S. Pat. No. 6,108,577, entitled, “METHOD AND APPARATUS FOR DETECTING CHANGES IN ELECTROCARDIOGRAM SIGNALS,” and U.S. patent application Ser. No. 09/962,852, entitled “EVOKED RESPONSE SENSING FOR ISCHEMIA DETECTION,” filed on Sep. 25, 2001, both assigned to Cardiac Pacemakers, Inc., which are incorporated herein by reference in their entirety.

In another embodiment, ischemia detector 332 detects the ischemic events from one or more impedance parameters. Sensor 330 includes an impedance sensing circuit to sense one or more impedance parameters each indicative of a cardiac impedance or a transthoracic impedance. Ischemia detector 332 includes an electrical impedance based sensor using a low carrier frequency to detect the ischemic events from electrical impedance. Tissue electrical impedance has been shown to increase significantly during ischemia and decrease significantly after ischemia, as discussed in Dzwonczyk, et al. IEEE Trans. Biomed. Eng., 51(12): 2206-09 (2004). The ischemia detector senses low frequency electrical impedance between electrodes interposed in the heart, and detects the ischemia as abrupt changes in impedance (such as abrupt increases in value). In a specific embodiment, ischemia detector 332 monitors complex impedance with concentration on the reactance to detect the ischemic events. Because ischemia induced changes in impedance occur predominantly in the reactive component, concentrating on the reactive component of the impedance provides for a high sensitivity of ischemia detection. In another specific embodiment, ischemia detector 332 detects the ischemic events from multiple impedance parameters sensed through multiple electrodes positioned to monitor ventricular regional volumes or wall motion. The impedance parameters are indicative of changes in regional cardiac contractions resulting from ischemia. The ischemic events are detected by analyzing morphological and/or timing changes in the impedance parameters, such as by using a template matching technique.

In another embodiment, ischemia detector 332 detects the ischemic events from one or more parameters indicative of heart sounds. Sensor 330 includes a heart sound sensing circuit. The heart sound sensing circuit senses the one or more parameters indicative of heart sounds using one or more sensors such as accelerometers and/or microphones. Ischemia detector 332 detects the ischemic event by detecting predetermined type heart sounds, predetermined type heart sound components, predetermined type morphological characteristics of heart sounds, or other characteristics of heart sounds indicative of ischemia.

In another embodiment, ischemia detector 332 detects the ischemic events from one or more pressure parameters. Sensor 330 includes a pressure sensing circuit coupled to one or more pressure sensors. In a specific embodiment, the pressure sensor is an implantable pressure sensor sensing a parameter indicative of an intracardiac or intravascular pressure whose characteristics are indicative of ischemia.

In another embodiment, ischemia detector 332 detects the ischemic event from one or more acceleration parameters each indicative of regional cardiac wall motion. Sensor 330 includes a cardiac motion sensing circuit coupled to one or more accelerometers each incorporated into a portion of a lead positioned on or in the heart. Ischemia detector 332 detects ischemia as an abrupt decrease in the amplitude of local cardiac accelerations.

In another embodiment, ischemia detector 332 detects the ischemic event from a parameter indicative of heart rate variability (HRV). Sensor 330 includes an HRV sensing circuit to sense and produce a parameter which is representative of a HRV. HRV is the beat-to-beat variance in cardiac cycle length over a period of time. The HRV parameter includes any parameter being a measure of the HRV, including any qualitative expression of the beat-to-beat variance in cardiac cycle length over a period of time. In a specific embodiment, the HRV parameter includes the ratio of Low-Frequency (LF) HRV to High-Frequency (HF) HRV (LF/HF ratio). The LF HRV includes components of the HRV having frequencies between about 0.04 Hz and 0.15 Hz. The HF HRV includes components of the HRV having frequencies between about 0.15 Hz and 0.40 Hz. Ischemia detector 332 detects ischemia when the LF/HF ratio exceeds a predetermined threshold. An example of an LF/HF ratio-based ischemia detector is discussed in U.S. patent application Ser. No. 10/669,168, entitled “METHOD FOR ISCHEMIA DETECTION BY IMPLANTABLE CARDIAC DEVICE,” filed on Sep. 23, 2003, assigned to Cardiac Pacemakers, Inc., which is incorporated by reference in its entirety.

FIG. 4 is a block diagram showing an embodiment of a cardioprotective device 400 for delivering the device therapy. Device 400 is a specific embodiment of device 300 and includes sensor 330, ischemia detector 332, a cardioprotective therapy controller 440, and a cardioprotective therapy output device 450.

Cardioprotective therapy controller 440 is a specific embodiment of cardioprotective therapy controller 340 and initiates and controls the delivery of the one or more cardioprotective therapies. In one embodiment, as illustrated in FIG. 4, cardioprotective therapy controller 440 includes a neurostimulation controller 442, a pacing controller 444, a gene regulatory controller 446, and a drug delivery controller 448. In other embodiments, cardioprotective therapy controller 440 includes any one or more of neurostimulation controller 442, pacing controller 444, gene regulatory controller 446, and drug delivery controller 448.

Cardioprotective therapy output device 450 is a specific embodiment of cardioprotective therapy output device 350 and delivers the one or more cardioprotective therapies. In one embodiment, as illustrated in FIG. 4, cardioprotective therapy output device 450 includes a neurostimulation output circuit 452, a pacing output circuit 454, a gene regulatory signal delivery device 456, and a drug delivery device 458. In other embodiments, cardioprotective therapy output device 450 includes any one or more of neurostimulation output circuit 452, pacing output circuit 454, gene regulatory signal delivery device 456, and drug delivery device 458.

Neurostimulation controller 442 controls a neurostimulation therapy that is delivered by neurostimulation output circuit 452. In one embodiment, the neurostimulation therapy includes the VST in which electrical stimulation pulses are delivered to the vagus nerve (see, e.g., Fallen et al., A.N.E., 10:441 (2005)).

Examples of neural stimulation can be found in the following patent applications which are herein incorporated by reference in their entirety: U.S. application Ser. No. 11/468,143, filed Aug. 29, 2006, and entitled “Controlled Titration of Neurostimulation Therapy,” and in U.S. application Ser. No. 11/468,135, filed Aug. 29, 2006, and entitled “System and Method For Neural Stimulation.” Various embodiments deliver the neural stimulation to a vagus nerve using a nerve cuff electrode and a lead subcutaneously tunneled from the device to the nerve. Various embodiments deliver the neural stimulation to a vagus nerve using a lead intravascularly fed to place at least one electrode in the internal jugular vein or other vessel proximate to the vagal target.

By way of example and not limitation, in one embodiment is, neural stimulation is delivered as electrical pulses at a frequency of approximately 20 Hz, a pulse width of approximately 300 microseconds, and a pulse amplitude of approximately 1.5 to 2.0 milliamps. The electrical pulses may each have a biphasic or monophasic waveform. The neural stimulation can be delivered as a pulse train applied either continuously or intermittently (e.g., with a duty cycle=10 seconds ON, 50 seconds OFF) in order to obtain a desired neural response. Such stimulation may be applied either chronically or periodically in accordance with lapsed time intervals or sensed physiological conditions. Various stimulation electrode configurations can be used, including, for example, a bipolar configuration with two electrodes on or near the target nerve, or a unipolar configuration with an electrode on or near the target nerve and a far-field subcutaneous return electrode. The stimulation circuitry may be either dedicated to delivering neural stimulation or may be configured to also deliver waveforms suitable for cardiac stimulation such as pacing and cardioversion/defibrillation.

The neural stimulation may be delivered as biphasic or monophasic pulse trains. In an embodiment, a neural stimulation waveform is delivered with phases of alternating polarity. For example, the waveform may be delivered as monophasic pulses with a bipolar stimulating configuration and with a “bipolar switch” so that the phase of the monophasic pulses is alternated in each consecutive pulse train. That is, a pulse train with monophasic pulses having first phases of one polarity is then followed by a pulse train with monophasic pulses having second phases of the opposite polarity.

The circuitry can be adapted to output pulses at a stimulation intensity specified by the controller. Various embodiments provide the ability to modulate the neural stimulation therapy by modulating feature(s) of the neural stimulation waveform such as amplitude, frequency, pulse width, pulse train duration, and the like.

Pacing controller 444 controls delivery of cardiac pacing pulses from pacing output circuit 454. In one embodiment, the pacing therapy includes a cardioprotective pacing therapy. The cardioprotective pacing therapy includes delivery of alternating pacing and non-pacing periods. Pacing controller 444 initiates and times one or more cardioprotective pacing sequences in response to the detection of the ischemic event by ischemia detector 332. The one or more cardioprotective pacing sequences each include alternating pacing and non-pacing periods. The pacing periods each have a pacing duration during which a plurality of pacing pulse is delivered. The non-pacing periods each have a non-pacing duration during which no pacing pulse is delivered. The one or more cardioprotective pacing sequences each have a sequence duration in a range of approximately 30 seconds to 1 hour. The pacing duration is in a range of approximately 5 seconds to 10 minutes. The non-pacing duration is in a range of approximately 5 seconds to 10 minutes. In one embodiment, the function of cardioprotective pacing is included in a pacing device that delivers pacing therapies on a long-term basis, such as for treatment of bradycardia and heart failure. The cardioprotective pacing therapy is a temporary pacing therapy delivered for one or more brief periods in response to the detection of the ischemia event, and the pacing device also delivers a chronic pacing therapy such as a bradycardia pacing therapy, a cardiac resynchronization therapy, and a cardiac remodeling control therapy. The temporary pacing therapy uses a pacing mode that is substantially different from the pacing mode of the chronic pacing therapy, such that the cardioprotective pacing therapy changes the distribution of stress in the myocardium, thereby triggering the intrinsic myocardial protective mechanism against ischemic damage to the myocardial tissue. Examples of the temporary pacing mode include VOO, VVI, VDD, and DDD modes, including their rate-responsive versions if applicable. In one embodiment, the pacing rate is set to be about 20 pulses per minute higher than the patient's intrinsic heart rate during the temporary pacing mode. In a specific embodiment, if the cardioprotective pacing therapy is the only pacing therapy being delivered (in other words, the chronic pacing mode is a non-pacing mode), the temporary pacing mode is an atrial tracking pacing mode such as the VDD or DDD mode, including their rate-responsive and multi-ventricular site versions. If the chronic pacing mode is an atrial tracking pacing mode such as the VDD or DDD mode, the temporary pacing mode is a VOO or VVI mode at with a pacing rate higher than the patient's intrinsic heart rate or a VDD or DDD mode with substantially different pacing parameter such as a pacing rate, pacing sites, and/or atrioventricular pacing delays. An example of an implantable medical device that delivers cardioprotective pacing (also referred to as cardiac protection pacing) is discussed in U.S. patent application Ser. No. 11/382,849, entitled “METHOD AND APPARATUS FOR INITIATING AND DELIVERING CARDIAC PROTECTION PACING,” filed on May 11, 2006, assigned to Cardiac Pacemakers, Inc., which is incorporated by reference in its entirety. In one embodiment, the cardioprotective pacing is delivered during a revascularization procedure, such as an angioplasty procedure, through pacing electrodes incorporated onto a percutaneous transluminal vascular intervention device. An example of a system for delivering cardioprotective pacing during a revascularization procedure, including percutaneous transluminal vascular intervention device with pacing electrodes, is discussed in U.S. patent application Ser. No. 11/113,828, entitled “METHOD AND APPARATUS FOR PACING DURING REVASCULARIZATION,” filed on Apr. 25, 2005, assigned to Cardiac Pacemakers, Inc., which is incorporated by reference in its entirety.

Gene regulatory controller 446 controls a gene therapy that is regulated by a gene regulatory signal delivered by gene regulatory signal delivery device 456. The gene regulatory signal is in a form of energy capable of regulating gene expression in a gene therapy vector. The forms of energy include electrical energy, electromagnetic energy, optical energy, acoustic energy, thermal energy, and any other form of energy that regulates expression in a gene therapy vector. Examples of the gene regulatory signal include an electric field, an electromagnetic field, a light, an acoustic signal such as a sound or an ultrasound signal, a chemical agent, and a thermal signal. In one embodiment, gene regulatory signal delivery device 456 delivers the gene regulatory signal to blood. In another embodiment, gene regulatory signal delivery device 456 delivers the gene regulatory signal to the heart. The gene regulatory signal is capable of regulating gene expression without inducing cardiac depolarization. In one embodiment, gene regulatory controller 446 initiates a biologic therapy in response to the detection of the ischemic event by ischemia detector 332. The biologic therapy includes alternating signaling and non-signaling periods. Each signaling period has a signaling duration during which the gene regulatory signal is emitted. Each non-signaling period has a non-signaling duration during which no gene regulatory signal is emitted. The biologic therapy has a therapy duration in a range of approximately 30 seconds to 1 hour. The signaling duration is in a range of approximately 5 seconds to 10 minutes. The non-signaling duration is in a range of approximately 5 seconds to 10 minutes. An example of a system for delivering such a biologic therapy is discussed in U.S. patent application Ser. No. 11/220,397, entitled “METHOD AND APPARATUS FOR DEVICE CONTROLLED GENE EXPRESSION FOR CARDIAC PROTECTION,” filed on Sep. 6, 2005, assigned to Cardiac Pacemakers, Inc., which is incorporated by reference in its entirety.

Drug delivery controller 448 controls the delivery of a cardioprotective agent from drug delivery device 458. Examples of a cardioprotective agent include but are not limited to beta-blockers, diuretics, ACE inhibitors, calcium channel blockers, nitrates, antiplatelets (anticlotting agents), and the like. In one embodiment, the agent is one employed for preconditioning or postconditioning, including but not limited to adenosine, bradykinin, acetylcholine and heat shock protein. In another embodiment, the agent lowers the pH of the myocardium, thereby providing postconditioning cardioprotection.

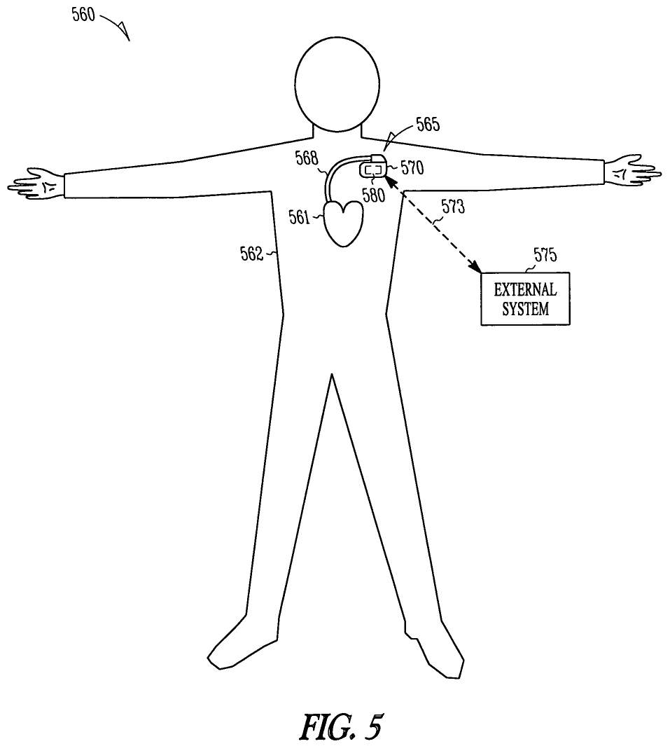

FIG. 5 is an illustration of an embodiment of a cardiac rhythm management (CRM) system 560 and portions of an environment in which system 560 operates. System 560 includes an implantable system 565, an external system 575, and a telemetry link 573 providing for communication between implantable system 565 and external system 575.

Implantable system 565 includes, among other things, implantable medical device 570 and lead system 568. In various embodiments, implantable medical device 570 is an implantable CRM device including one or more of a pacemaker, a cardioverter/defibrillator, a cardiac resynchronization therapy (CRT) device, a cardiac remodeling control therapy (RCT) device, a neruostimulator, a drug delivery device or a drug delivery controller, and a biological therapy device. As illustrated in FIG. 5, implantable medical device 570 is implanted in a body 562. In various embodiments, lead system 568 includes leads for sensing physiological signals and delivering pacing pulses, cardioversion/defibrillation shocks, neurostimulation pulses, pharmaceutical agents, biological agents, and/or other types of energy or substance for treating cardiac disorders. In one embodiment, lead system 568 includes one or more pacing-sensing leads each including at least one electrode placed in or on a heart 561 for sensing electrogram and/or delivering pacing pulses. In other embodiments, electrodes placed in body 562 but away from heart 561 are used to sense physiological signals and deliver pacing pulses, cardioversion/defibrillation shocks, neurostimulation pulses, pharmaceutical agents, biological agents, and/or other types of energy or substance for treating cardiac disorders. In a specific embodiment, one or more electrodes are incorporated onto implantable medical device 570 for subcutaneous placement.

System 560 includes cardioprotective device 300 or 400. Implantable medical device 570 includes a cardioprotective device 580, which includes cardioprotective device 300 or 400, or portions thereof. In one embodiment, lead system 568 represents an interface between cardioprotective therapy output device 350 or 450 and body 562. One or more leads may include electrodes for delivering one or more of neurostimulation, pacing pulses, and an electrical gene regulatory signal and/or an agent delivery structure for delivering one or more of gene regulatory and cardioprotective agents.

External system 575 allows a physician or other caregiver or a patient to control the operation of implantable medical device 570 and obtain information acquired by implantable medical device 570. In one embodiment, external system 575 includes a programmer communicating with implantable medical device 570 bi-directionally via telemetry link 573. In another embodiment, external system 575 is a patient management system including an external device communicating with a remote device through a telecommunication network. The external device is within the vicinity of implantable medical device 570 and communicates with implantable medical device 570 bi-directionally via telemetry link 573. The remote device allows the physician or other caregiver to monitor and treat a patient from a distant location. In one embodiment, an ischemia alert signal is produced in implantable medical device upon detection of an ischemia event and transmitted to the remote device for producing an alarm signal and/or a warning message for the physician or other caregiver.

Telemetry link 573 provides for data transmission from implantable medical device 570 to external system 575. This includes, for example, transmitting real-time physiological data acquired by implantable medical device 570, extracting physiological data acquired by and stored in implantable medical device 570, extracting therapy history data stored in implantable medical device 570, transmitting signals generated by implantable medical device 570 such as the ischemic alert signal, and extracting data indicating an operational status of implantable medical device 570 (e.g., battery status and lead impedance). Telemetry link 573 also provides for data transmission from external system 575 to implantable medical device 570. This includes, for example, programming implantable medical device 570 to acquire physiological data, programming implantable medical device 570 to perform at least one self-diagnostic test (such as for a device operational status), and programming implantable medical device 570 to deliver at least one therapy, such as to initiate a cardioprotective therapy.

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

1. A vector system, comprising:
a) a first vector comprising a transcriptional regulatory region which includes an organ-specific, tissue-specific or cell-specific, or energy-regulated, transcriptional regulatory element operably linked to a first open reading frame for a gene product that is unstable under normoxic conditions, stable under hypoxic conditions and binds a specific DNA sequence; and
b) a second vector comprising a transcriptional regulatory region comprising the specific DNA sequence operably linked to a second open reading frame for a therapeutic gene product, cardioprotective gene product or biomarker.

2. The vector system of claim 1 wherein the transcriptional regulatory region in the first vector comprises a cardiac-specific transcriptional regulatory element.

3. The vector system of claim 1 wherein the first open reading frame encodes a hypoxia induced gene product.

4. The vector system of claim 1 wherein the second open reading frame encodes a receptor.

5. The vector system of claim 4 wherein the second open reading frame encodes an acetylcholine receptor (AChR).

6. The vector system of claim 1 wherein the second open reading frame encodes an endothelial nitric oxide synthase (eNOS).

7. The vector system of claim 1 wherein the second open reading frame encodes a hemeoxygenase (HO).

8. The vector system of claim 1 wherein the second open reading frame encodes a heat shock protein (HSP).

9. The vector system of claim 1 wherein the second open reading frame encodes Akt.

10. The vector system of claim 1 wherein the second open reading frame encodes a secreted protein.

11. The vector system of claim 10 wherein the second open reading frame encodes secreted alkaline phosphatase (SEAP).

12. The vector system of claim 1 wherein the second open reading frame encodes a nonvertebrate cell associated biomarker.

13. The vector system of claim 1 wherein the second open reading frame encodes a growth factor.

14. The vector system of claim 13 wherein the second open reading frame encodes vascular endothelial growth factor (VEGF).

15. The vector system of claim 1 wherein the second open reading frame encodes a cytokine.

16. The vector system of claim 1 wherein the second open reading frame encodes an anchor protein.

17. The vector system of claim 16 wherein the second open reading frame encodes a sodium iodide symporter (NIS).

18. The vector system of claim 1 wherein the expression of the second open reading frame in an effective amount in cells of a mammal prevents, inhibits or treats cardiac ischemia in the mammal.

19. A mammalian cell having the vector system of claim 1.

20. The mammalian cell of claim 19 which is a stem cell.

21. A system, comprising;
a composition comprising at least two vectors, wherein a first vector comprises a transcriptional regulatory region which includes an organ-, tissue- or cell-specific, or energy-regulated, transcriptional regulatory element operably linked to a first open reading frame for a gene product that is unstable under normoxic conditions, stable under hypoxic conditions and binds a specific DNA sequence, and wherein a second vector comprises a transcriptional regulatory region comprising the specific DNA sequence linked to a second opening reading frame for a therapeutic gene product or a cardioprotective gene product; and
an implantable device adapted to deliver one or more of electrical, biologic, or drug therapy which enhances the efficacy of the therapeutic or cardioprotective gene product.

22. The system of claim 21 wherein the tissue-specific transcriptional regulatory element is cardiac-specific.

23. The system of claim 21 wherein the first open reading frame encodes hypoxia inducible factor.

24. The system of claim 21 wherein the first open reading frame encodes cAMP regulatory element binding protein.

25. The system of claim 21 wherein the implantable device comprises an ischemia detector to detect an ischemia event.

26. The system of claim 25 wherein the implantable device comprises a sensor to sense a secreted alkaline phosphatase (SEAP) level, and the ischemia detector is adapted to detect the ischemia event using the SEAP level.

27. The system of claim 21 wherein the implantable device comprises a neurostimulator.

28. The system of claim 21 wherein the implantable device comprises a cardiac pacemaker.

29. The system of claim 21 wherein the implantable device comprises a gene regulatory signal delivery device.

30. The system of claim 21 wherein the implantable device comprises a drug delivery device.

31. The system of claim 21 wherein the first and second vectors are in donor mammalian cells.

32. The system of claim 31 wherein the first and second vectors are in mammalian cardiac cells.

33. The system of claim 31 wherein the first and second vectors are in mammalian stem cells.

34. A method to detect ischemia, comprising:
a) providing a mammal having cells comprising the vector system of claim 1, wherein the second open reading frame encodes a secretable biomarker or a gene product which binds sodium, phosphorus, iodine, carbon, or gadolinium, or an analog thereof; and
b) detecting the secretable biomarker or the protein which binds sodium, phosphorus, iodine, carbon, or gadolinium, or an analog thereof in the mammal.

35. The method of claim 34 further comprising delivering to the mammal electrical therapy in response to detection of the secretable biomarker.

36. The method of claim 34 wherein the secretable biomarker comprises SEAP.

37. The method of claim 34 wherein an implantable device detects the secreted biomarker.

38. The method of claim 34 wherein the location or presence of the protein which binds sodium, phosphorus, iodine, carbon, or gadolinium, or an analog thereof is detected by administering radioactive sodium, phosphorus, iodine, carbon, or gadolinium, or an analog thereof, or a molecule containing radioactive sodium, phosphorus, iodine, carbon, or gadolinium, or an analog thereof, or gadolinium.

39. A method to treat ischemia, comprising introducing the vector system of claim 1 to a mammal, wherein the second open reading frame encodes a therapeutic or cardioprotective gene product.
